(19) United States
(12) Patent Application Publication
(10) Pub. No.: US 2002/0111323 A1
(43) Pub. Date: Aug. 15, 2002

(54) Introduction of naked DNA or RNA encoding non-human vertebrate peptide hormones or cytokines into a non-human vertebrate

(76) Inventors: Stephen Martin, Portage, MI (US); Paul F. Russell, Portage, MI (US)

Correspondence Address: Edward F. Rehberg, Pharmacia & Upjohn Company, Global Intellectual Property, 301 Henrietta Street, Kalamazoo, MI, 49001, US

(21) Appl. No.: 09/898,428

(22) Filed: Jul. 2, 2001

Related U.S. Application Data
(63) Continuation of application No. 09/292,188, filed on Apr. 15, 1999, now abandoned.
(60) Provisional application No. 60/084,418, filed on May 6, 1998.

(51) Int. Cl. A61K 48/00; A61K 9/127; A61K 38/19

(52) U.S. Cl. 514/44; 424/450; 424/85.1

(57) ABSTRACT

The present invention relates to the introduction of naked DNA or RNA molecules encoding non-human vertebrate peptide hormones or cytokines into a non-human vertebrate to achieve delivery of the non-human vertebrate peptide hormone or cytokine. The invention thus provides an alternative to directly administering the polypeptide of interest.

### CROSS REFERENCE TO RELATED APPLICATIONS

The present application is a continuation application of U.S. Ser. No. 09/292,188 filed on Apr. 15, 1999 which is a continuation-in-part of U.S. Ser. No. 60/084,418 filed May 6, 1998.

### BACKGROUND OF THE INVENTION

1. Field of the Invention

The present invention relates to the introduction of naked DNA or RNA molecules encoding non-human vertebrate peptide hormones or cytokines into a non-human vertebrate to achieve delivery of the non-human vertebrate peptide hormone or cytokine. The invention thus provides an alternative to directly administering the polypeptide of interest.

2. Related Art

Somatic growth is under the influence of multiple hormones, however, the primary regulator is growth hormone (GH) or somatotropin (ST). GH is secreted in a pulsatile fashion from the anteroir pituitary gland. In humans GH sevretion occurs primarily during the third and fourth stages of slow wave sleep. The secretion of GH is controlled predominantly by the hypothalamic hormones, growth hormone releasing hormone (GHRH) and somatostatin, that increase and decrease the secretion of GH respectively. In the plasma a significant portion (10-50%) of the GH is bound to a 60kDa binding protein that is identical to the extracellular binding domain of the GH cellular receptor. GH acts upon it's target tissues through a dimerization of the cellular receptors, that in turn induces the production of the somatomedins, primarily insulin like gowth factor type 1 (IGF1). Similarly to GH the IGF's are bound to plasma proteins (primarily IGFBP3) that are thought to increase their circulatory half lives. The IGF's mediate many of the anabolic effects of GH in the target tissues through increased cellular proliferation and retention of amino acids. In addition the IGF's also negatively feedback to the anterior pituitary to inhibit further GH secretion.

The descriptions of the results of exogenous application of GH upon humans (GH deficient) and animals (primarily food animal species) are consistent with its anabolic potential. Thus increases in normal GH levels lead to an increased bone growth and mass, increased lean muscle mass, and decreased adipose tissue. The effects of administration of recombinant porcine growth hormone (pST) or GHRH upon performance in swine has been studied at PNU and reported in the literature. If sufficient GH is given consistently to growing animals (by daily injection, for example) then the end result is an increase in the rate and efficiency of lean body weight gain. If sufficient exogenous GH is applied consistently to lactating cows (by daily injection, for example) then its galactogenic effects become evident as an increase in milk yield. In all of the examples cited so far it has been necessary to give large amounts of GH over sustained periods in order to see the anabolic or galactogenic effect. One reason for this is that the large exogenous amounts of GH will cause the negative effects of the GH cascade (somatostatin and IGF1) to minimize the production of endogenous GH by the anterior pituitary. Consequently, the exogenous GH applied must be sufficient to not only replace the normal daily GH levels produced by the anterior pituitary but to also elevate the GH levels above those normally present in the animal.

In addition to its anabolic effects, GH is also reported in the literature to have positive effects upon the immune system. GH and IGF receptors have been identified upon lymphocytes and cells of the reticulo-endothelial system. In vitro studies have demonstrated that both GH and IGF-1 can elevate the proliferation of lymphocytes after stimulation. GH has been shown to an essential element for the maturation of thymocytes into mature T lymphocytes an also to protect animals from endotoxic shock after exposure to lipopolysaccharides.

Genetic immunization is the direct inoculation of bacterial plasmids into tissues of a mammalian host. When these bacterial plasmids contain a eukaryotic expression cassette, the gene product will be expressed and lead to one of several biologcal effects. If the encoded gene product is foreign to the host one consequence of the expression will be the induction of an immune response. Genetic immunization has been used very successfully to induce antibody and cytotoxic T lymphocyte responses to the gene products of a broad spectrum of potentially pathogenic microorganisms. Other biological consequences of the expressed product will depend upon if the gene product is an enzyme of hormone. The expressed product will then act upon its normal physiological target that is present within the host.

There are few reports upon the use of genetic immunization to deliver biological active molecules, but ameliorating effects upon vascular hypertension have been described for cDNA encoding kallikrein. Although plasmid DNA administered via genetic immunization is long lived in the host, it has not been demonstrated to express very significant levels of the encoded gene product in vivo. Consequently, in a biological system such as the GH cascde where increased levels of the meditor due to exogenous supply (for example GH) leads to a decrease in physiological production, genetic immunizatio may not produce enough protein to both replace the endogenous levels and increase levels above the normal physiological norm.

The use of naked DNA (bacterial plasmids containing a eukaryotic expression cassette encoding a protein of interest) to immunize animals has been used with considerable success by many investigators around the world for the last few years. The preferred delivery technique is either intramuscular injection of a DNA solution or the ballistic delivery of gold particle-coated DNA into the dermis of an animal. We have had preliminary successes immunizing either mice or swine with naked DNA encoding virus glycoproteins, cytokines or bovine growth hormone (bST). However, very few outside researchers or ourselves have been able to conclusively demonstrate that cytokines or growth hormones delivered by this mechanism can achieve high enough serum levels within the host to induce an appropriate biological response (for example performance enhancement due to exogenous growth hormone). Earlier experiments this year in swine injected with plasmids encoding bST, demonstrated that sufficient bovine growth hormone was produced to induce high titer anti-bST antibodies in the serum of about 50% of immunized animals. Since small increases in serum levels of endogenous somatotropins due to exogenous administration of the protein, increased growth rate in pigs fed adequate amounts of crude protein, we decided to use a well characterized and sensitive growth screen model to determine if i.m. injection of naked plasmid pST-DNA can improve average daily gain (ADG) and feed efficiency (FE) of pigs.

Direct inoculation of animals with bacterial plasmids encoding a eukaryotic expression cassette has been shown to be an effective means of generating an immune response against a wide array of protein antigens (Rev 1). Purified plasmid DNA can be used to inoculate tissues by simple injection in a saline solution or by ballistic delivery of DNA precipitated onto small inert (gold) beads. Following either type of delivery the predominant cell type surrounded the inoculation site is usually transfected and expresses the gene product encoded by the eukaryotic expression cassette. In the case of needle delivery the usual route of inoculation is intramuscularly and the muscle cell is the predominantly transfected cell. The method of transfection is controversial, but it appears that muscle cells will actively take up the injected DNA from the extracellular environment (2-4). In contrast ballistic delivery targets the DNA to the epidermis and the predominant cell type is the keratinocyte (5). Ballistic delivery is much more efficient than needle delivery, requiring 100 to 1000 fold less DNA, presumably because the DNA is propelled directly into the cytoplasm of the keratinocytes (6,7). Another significant difference between these two methods of DNA immunization reflects the in vivo half lives of the primary cell type transfected. Plasmid DNA inoculated into muscle tissue is still detectable and remains transcriptionally active for periods of one year and longer (8-9). Whereas ballistically delivered DNA is mostly lost within a few weeks of inoculation due to the natural desquamation process of the host's dermal layers.

Most of the descriptions of the application of DNA immunization have focused on it's ability to induce antibody (Ab) and cytotoxic T lymphocyte (Tc) against the encoded protein (rev 1). Interestingly the induction of these immune responses are largely independent of the cells transfected around the inoculation site and is largely dependent upon the somatic cells derived from the host's bone marrow. This implies that the transfected cells responsible for inducing the immune response are antigen presenting cells (apc) transfected at the site of inoculation (10). A more likely or contributing effect will be that some of the DNA is directed to lymphoid tissue draining the site of inoculation and transfects long lived, potent apc such as dendritic cells there.

The use of DNA immunization as a sustained delivery vehicle for modulatory proteins such as hormones and cytokines has not been described by many researchers. The few successful reports in the literature include increased serum levels of apolipoprotein A in rats (11), the down modulation of herpetic stromal keratitis by inoculation of plasmids encoding murine interleukin 10 into the cornea of infected mice (12), and the expression of the kallikrein gene as therapy for hypertension in cardiovascular and renal disease (13). Given our initial successes using DNA immunizations to successfully vaccinate swine and rodents, we decided to investigate this technique as a delivery method for bovine growth hormone.

Preferred proteins or peptides for the incorporation into the compositions of the invention are insulin and insulin-like growth factors, interferon, growth hormone releasing factor, interleukins, etc. Most preferred are the growth hormones or somatotropins, especially bovine and porcine somatotropin, and growth hormone releasing factor. The protein or peptide may be obtained from the natural tissue (“native”) or produced by recombinant technology (“recombinant”) and includes proteins or peptides having modified or varied amino acid sequences. The essential feature is that the protein or peptide retain bioactivity in the species into which it is administered.

### SUMMARY OF THE INVENTION

The present invention provides a method for the introduction of naked DNA or RNA molecules encoding non-human vertebrate peptide hormones or cytokines into a non-human vertebrate to achieve delivery of the non-human vertebrate cytokine.

In one embodiment, the invention relates to a method for delivering a desired physiologically active protein, polypeptide or peptide growth hormone or cytokine to a non-human vertebrate, comprising injecting into the muscle of said vertebrate a non-infectious, non-immunogenic, non-integrating DNA sequence encoding said growth hormone or cytokine operably linked to a promoter, wherein said DNA sequence is free from association with transfection-facilitating proteins, viral particles, liposomal formulations, charged lipids and calcium phosphate precipitating agents, whereby said DNA sequence is expressed.

In a preferred embodiment, the vertebrate is a mammal.

In another preferred embodiment, the growth hormone or cytokine is selected from the group consisting of porcine growth hormone, bovine growth hormone, canine growth hormone, bovine IGF-1, porcine IGF-1, canine IGF-1, bovine growth hormone releasing factor, porcine growth hormone releasing factor, and canine growth hormone releasing factor. In a more highly preferred embodiment, the growth hormone or cytokine has an amino acid sequence identical to the native growth hormone or cytokine of said vertebrate.

### BRIEF DESCRIPTION OF THE FIGURES

FIG. 1 shows the construction of plasmids p3CIa and p3CIag.

FIG. 2 shows the construction of plasmids p3CLb and p3CLbg.

FIG. 3 is a map of plasmids generated by the insertion of cytokine genes into p3CIa and p3CLb.

FIG. 4 is a map of plasmids generated by the insertion of cytokine genes into p3CIag and p3CLbg.

FIG. 5 is a graph showing the quantity of IFNg detected in adouble sandwich ELISA.Vero cells were transfected with 1 mg of plasmid using different amounts of the LipofectAMINE (GibcoBRL). Cell supernatants were collected after 48 and 72 hours and IFNg was detected using a monoclonal antibody in a double sandwich ELISA. IFNg was detected in all transfections. Much less of the fusion proteins was detected. However, it is unclear if this represents less gIII-IFNg or simply poorer detection due to the chimeric nature of the protein.

FIG. 6 is a graph showing the inhibition of PRV infection of PK15 cells by pre treatment with either culture supernatants or purified gIFN.

FIG. 7 is a graph showing the effect of muscle pretreatment, amount of DNA, and presence of an intron in the construct on gene expression. This was measured by detection of anti-gIII antibody using an indirect ELISA.

FIG. 8 is a graph showing the effect of injection technique used to deliver 1 mg of plasmid DNA to swine. Number, depth, and volume of injections was examined with animals being boosted at 3 weeks. Detection of PRV specific antibody at 3 and 6 weeks after the initial injections was used to evaluate the various techniques.

FIG. 9 is a graph showing the effect of the amount of plasmid injected and the presence of an intron in the construct on gene expression in swine. Expression was measured by the detection of anti-gIII antibody via an indirect ELISA.

FIG. 10 is a graph showing the effect on swine of injection of plasmids carrying the porcine GM-CSF gene. Pigs received a single, 2 ml shot of either PBS or p3CIa/GM-CSF. Animals were bled daily and the numbered of PMN's and bands were determined. The wide variability between individuals does not allow for any statistically significant conclusions.

FIG. 11 is a graph showing the effect of injection with porcine IL-1υ DNA on temperature in swine. Two different gIII/IL-1υ constructs and their corresponding gIII parents were used. DNA pigs received a single, 2ml injection containing lmg DNA. Control pigs received PBS or no injection. Pigs were temped daily.

FIG. 12 is a graph showing the comparative levels of PRV gIIl specific antibodies in pigs from the IL-1B study. Antibody was detected using an indirect ELISA. Sera from prebleeds and final bleeds were tested for aIgM and aIgG. The presence of gIIl specific antibodies suggests that the genes of interest are being expressed in pigs following injection of plasmid DNA.

### DETAILED DESCRIPTION

The present invention relates to the introduction of naked DNA or RNA molecules encoding non-human vertebrate peptide hormones or cytokines into a non-human vertebrate to achieve delivery of the non-human vertebrate peptide hormone or cytokine.

In one embodiment, the invention relates to a method for delivering a desired physiologically active protein, polypeptide or peptide growth hormone or cytokine to a non-human vertebrate, comprising injecting into the muscle of said vertebrate a non-infectious non-immunogenic, non-integrating DNA sequence encoding said growth hormone or cytokine operably linked to a promoter, wherein said DNA sequence is free from association with transfection-facilitating proteins, viral particles, liposomal formulations, charged lipids and calcium phosphate precipitating agents, whereby said DNA sequence is expressed.

In a preferred embodiment, the vertebrate is a mammal, and the growth hormone or cytokine is selected from the group consisting of porcine growth hormone, bovine growth hormone, canine growth hormone, bovine IGF-1, porcine IGF-1, canine IGF-1, bovine growth hormone releasing factor, porcine growth hormone releasing factor, and canine growth hormone releasing factor. In a more highly preferred embodiment, the growth hormone or cytokine has an amino acid sequence identical to porcine growth hormone, bovine growth hormone, canine growth hormone, bovine IGF-1, porcine IGF-1, canine IGF-1, bovine growth hormone releasing factor, porcine growth hormone releasing factor, and canine growth hormone releasing factor. In a more highly preferred embodiment, the growth hormone or cytokine has an amino acid sequence identical to the native growth hormone or cytokine of said vertebrate.

The materials and methods necessary for practicing the claimed invention are described in U.S. Pat. No. 5,580,859, the contents of which are incorporated herein in their entirety, as well as in the following Examples.

Of course, one of ordinary skill in the art will readily be able to substitute DNA or RNA encoding the growth hormone or cytokine of interest into one of the vectors described in U.S. Pat. No. 5,580,859 or in the following Examples using well-established and routine techniques. Of course, where the amino acid sequence of the growth hormone or cytokine of interest is known, it will be well within the skill of an ordinary artisan to obtain DNA or RNA encoding it either by chemical synthesis, or by isolating the gene of interest from either a genomic or a cDNA library using a degenerate synthetic probe corresponding to a portion of the amino acid sequence.

The amino acid sequence of the following growth hormones, and in some cases the nucleotide sequence of the polynucleotide molecule encoding said growth hormones, may be found in the following publication, the contents of which are incorporated herein by reference in their entirety: porcine growth hormone: EP 0 104 920; canine growth hormone: DE 43 03 744; bovine growth hormone releasing factor: EP 0 212 531; porcine growth hormone releasing factor: Bvaskin et al., J. Animal Sci. (1997) 75(8):2285. As the amino acid sequences of porcine, bovine, and canine insulin-like growth factor-1 (IGF-1) are identical to that of human IGF-1 (Weller et al., Biochem. Genetics 120: 47105, 1994), it will be clear to the skilled artisan that knowledge of the amino acid sequence of human IGF-1 will enable one to synthesize a gene encoding IGF-1 from pig, cow, or dog. The amino acid sequence of human IGF-1 is disclosed in U.S. Pat. No. 5,070,075.

The following examples are provided by way of illustration and are not intended as limiting.

### EXAMPLE

### Example 1

### Preparation of Plasmids for the Expression of Porcine Cytokine Genes

Naked DNA technology may be used both for immunization (Donnelly, Ulmer et al. 1994; Hassett and Whitton 1996; Fazio 1997; Robinson, Ginsberg et al protein delivery system (Hengge, Chan et al. 1995; Wang, Chao et al. 1995; Lawson, Yeow et al. 1997). With the goal of using naked DNA technology to express regulatory molecules, such as cytokines, in swine, a series of plasmids designed to express the porcine cytokine genes for IL-2, IL-4, IL-10, IFNK, IL-1υ, IL-5, IL-6, and GM-CSF were constructed.

Materials and Methods

Materials: Plasmid DNA was isolated from E. coli bacteria using NaOH/SDS with subsequent purification by either CsCl gradient centrifugation or QIAGEN columns. Fragments were electroeluted from agarose gels and purified using NACS52 PREPAC columns (GIBco BRL). All restriction and modification enzymes were used according to the manufacturer's specifications.

Cytokine genes were provided by D. Strom and were received as BamHI/EcoRI fragments cloned into pUC-based vectors. Plasmid pSph2B9, containing the gIII gene from pseudorabics virus (PRV), was a gift of D. Thomsen.

PK15 and vero cells were grown in DMEM supplemented with 10% heat-inactivated fetal bovine serum. Transfections of PK15 and vero cells using LipofectAMINE Reagent (GIBco BRL) were performed according to the manufacturer's suggested protocol. The cells and culture supernatant were tested for expression of gIII and cytokines after 48 and/or 72 hours.

ELISA Methods: IFNK in cell supernatants was detected using a double sandwich ELISA, as described previously.

Virus Inhibition Assay: PK15 cells, resuspended at a density of 2.5×10⁵ cells per ml, were aliquoted, 2 ml per well, into six well plates and allowed to grow overnight. The next day, cells were washed with DMEM supplemented with 2% heat-inactivated fetal bovine serum. Cells were pretreated overnight with 750 ml of the same media plus 250 ml of culture supernatant from transiently expressing cultures. Negative supernatants came from mock transfections. Positive controls contained various amounts of purified, baculovirus-derived protein. Following pretreatment, cells were washed with media and then infected, for one hour, with dilutions of PRV designed to produce about 50 plaques per well. Wells were then overlaid with a Gibco medium 199 containing 1.5% LMP agarose and incubated for an additional 2 days. Visualization of virus plaques was done by overlaying with Medium 199 containing 1.5% LMP agarose and 0.01% phenol red. Plaques were visible in 5-6 hours.

Immunoperoxidase Staining Of Transfected Cell Monolayers: Vero cells were transfected as described above. After allowing for a period of expression, 48-72 hours, the cells were washed and fixed to the bottom of the plate. The plates were then treated with a primary antibody specific for the protein(s) of interest. Plates were washed and bound antibody was detected using an anti-species IgG conjugate labeled with HRPO. The substrate used for the final detection step was 3-amino-9-ethylcarbazole (AEC) in an acetate buffer containing hydrogen peroxide. Data was recorded photographically.

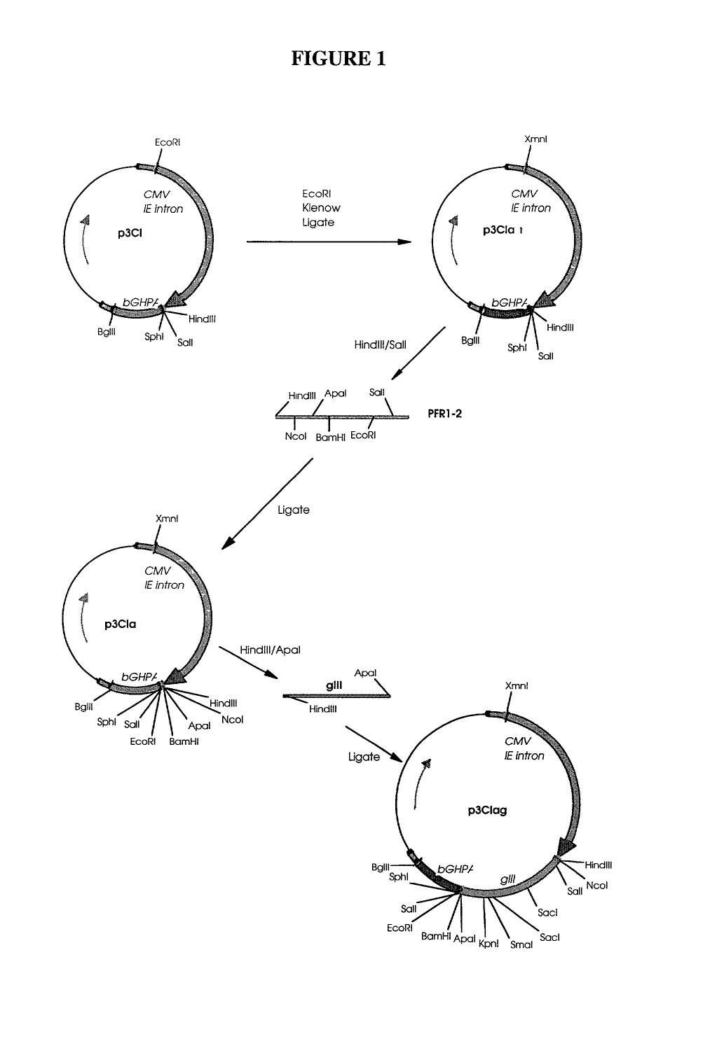

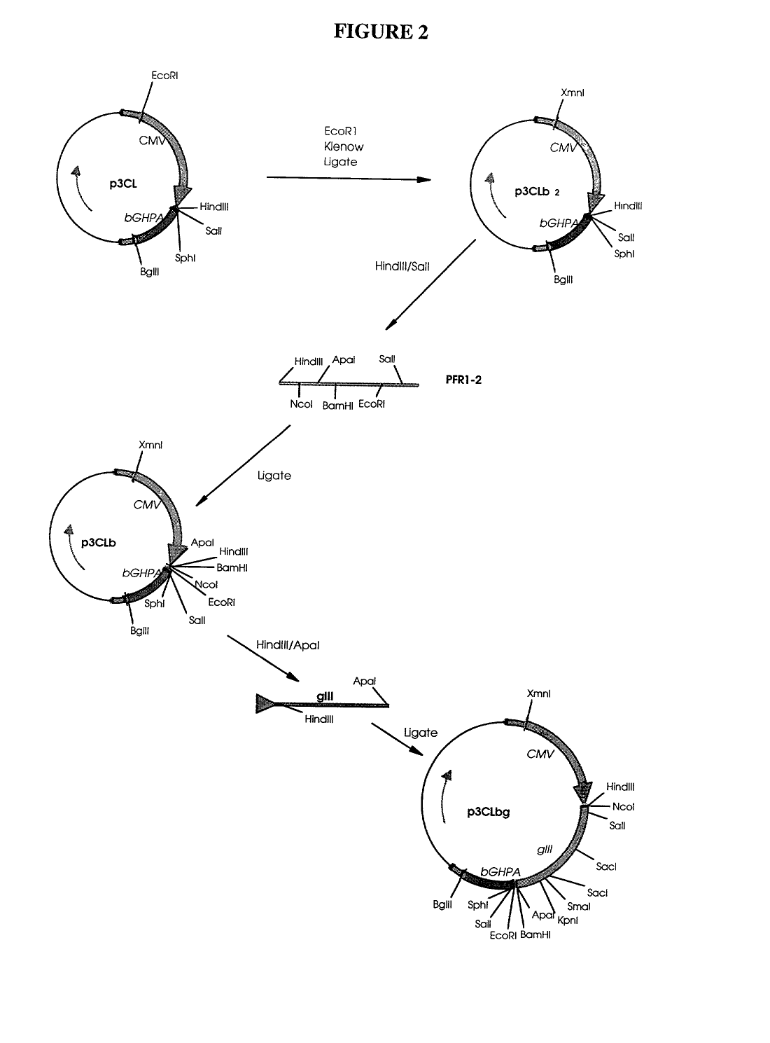

Plasmid Construction: The redesigning of plasmids p3CI and p3CL had two goals in mind. The first was to allow for easy cloning of several cytokine genes which were available as BamHI/EcoRl fragments. The second was to create a second generation vector which would express a partial PRV gIII protein fused in-frame with these cytokines (FIGS. 1 and 2). These goals were accomplished as follows.

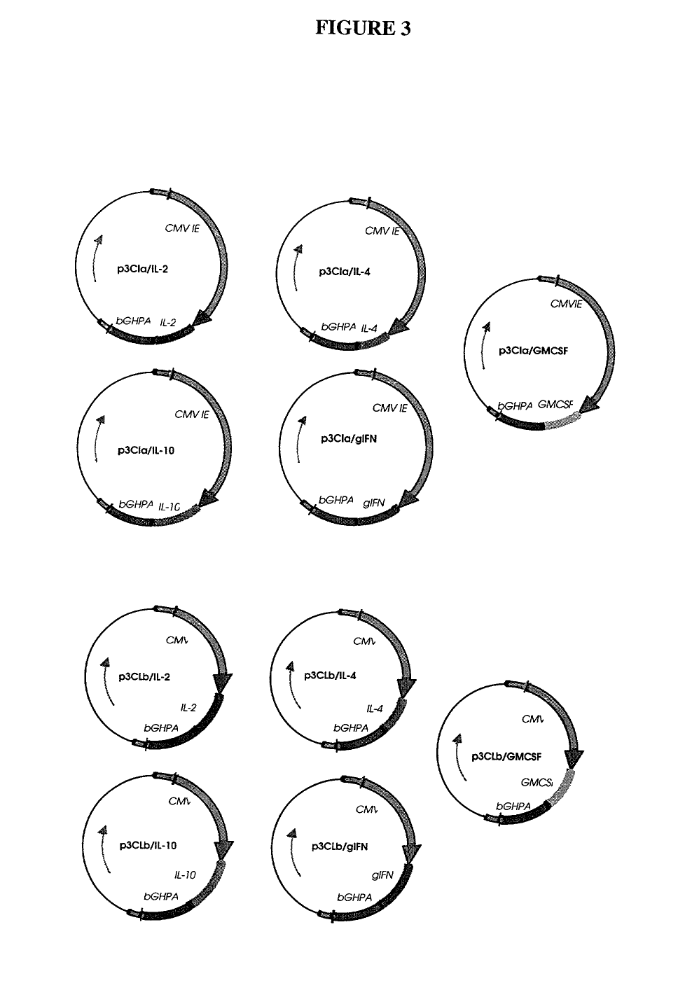

Plasmid p3CI was digested with EcoRI, end filled with Klenow, and religated. This removed the unique EcoRI site, 5′ to the CMV promoter, and replaced it with an XmnI site. This plasmid, p3CIa_{1,} was then digested with HindIII and SalI and ligated to the linker PFR1-2 to give the plasmid p3CIa. Plasmid p3CL was manipulated, as described above to produce p3CLb. These plasmids were suitable for cloning of the BamHI/EcoRI cytokine fragments (FIG. 3).

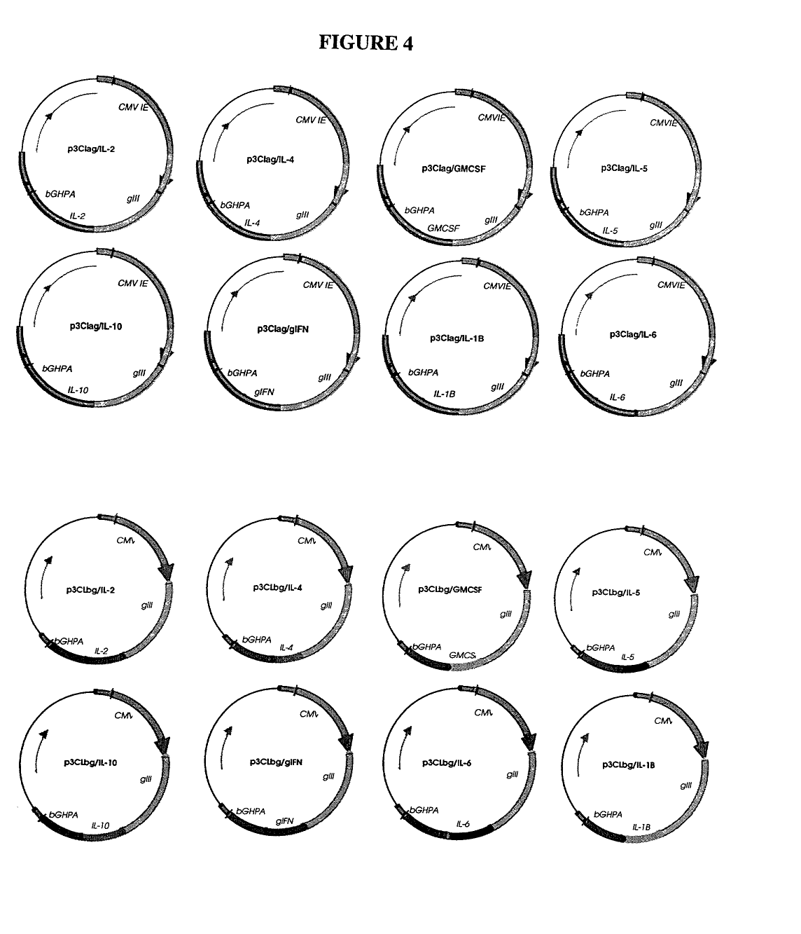

A HindIII/ApaI fragment comprising the entire PRV gIII gene, minus the transmembrane region, was then introduced into p3CIa and p3CLb to create the plasmids p3CIag and p3CLbg. BamHI/EcoRI cytokine fragments cloned into this plasmid would be expressed as gIII-cytokine fusion proteins (FIG. 4).

Immunostimulatory Motifs: Immunostimulatory, or CpG, motifs are short series of nucleotides generally following the formula 5′-Pur Pur CG Pyr Pyr-3′. When present in injected DNA, these motifs are reported to enhance the T_{H}1 response to the expressed gene product (Sun, Beard et al. 1977; Krieg, Yi et al. 1995; Krieg 1996; Pisetsky 1996; Yi, Chace et al. 1996; Klinman, Yamshchikov et al. 1997; Roman, Martin-Orozco et al. 1997). It has even been demonstrated that DNA lacking these CpG motifs failed to stimulate the typical T_{H}1 cytokine profile (Sato, Roman et al. 1996). We analyzed the sequences of p3CIa and p3CLb for the presence of immunostimulatory, or CpG, motifs. The analysis was performed using the Findpatterns program from the University of Wisconsin GCG package. Seven examples of these motifs were detected in each plasmid.

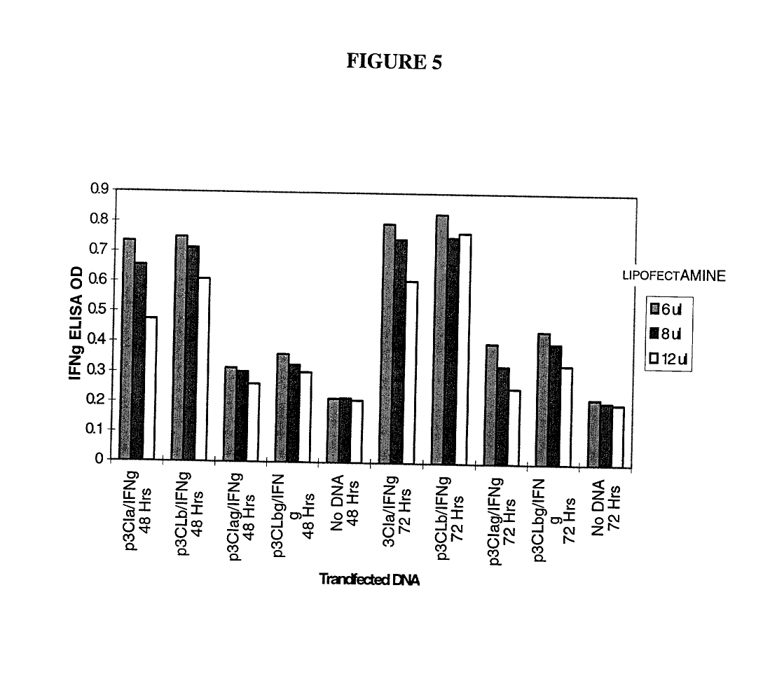

In Vitro Expression: Vero cells were transfected with p3CIa, p3CIa/IFNg, p3CIag, p3CIag/IFNg, p3CLb, p3CLb/IFNg, p3CLbg, and p3CLbg/IFNg using LipofectAMINE Reagent. Culture supernatants were tested, by ELISA, for expression of IFNg (FIG. 5). IFNg was easily detectable in culture supernatants from the p3CIa/IFNg and p3CLb/IFNg transfections. Results from transfections involving plasmids carrying the gIII-IFNg fusions were less dramatic.

The transfected cell monolayer was examined to determine if the lower levels of gIII-IFNg fusion protein, detected in the supernatants, was due to a lack of transport out of the cell. After the culture supernatant was removed from the plates, the cells were fixed and subjected to immunoperoxidase staining using monoclonal antibodies to gIII and IFNg. The results support the idea that gIII and the gIII-IFNg fusion are not as readily exported from the cells as IFNg alone.

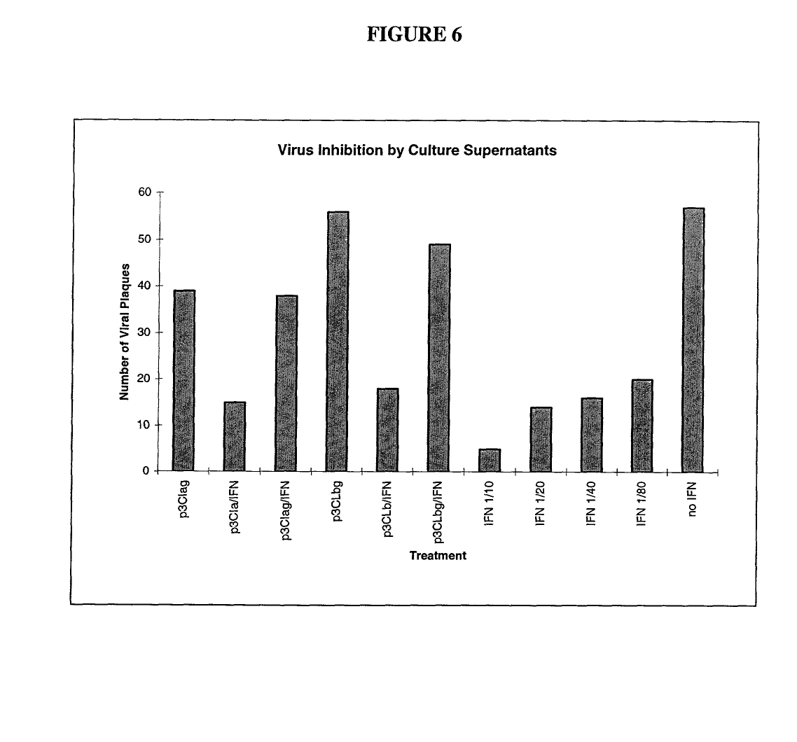

Having demonstrated expression of the gIII, IFNg, and gIII-IFNg genes, we wanted to test whether or not the gene products, produced in vitro, were biologically active. Supernatants from a transfection of PK15 cells were used in virus inhibition assays. Transfection supernatants produced by IFNg plasmids demonstrably reduced the number of PRV plaques when compared to mock transfection and purified IFNg treatments. gIII-IFNg fusion transfections, however, did not show any dramatic evidence of inhibition (FIG. 6). This could be attributed to the lower expression levels found with these constructs. It is also possible that the fusion protein is inherently less active than the native protein.

Additional transfections of vero cells were done with plasmids carrying the IL-2, IL-4, and IL-10 cytokine genes. Immunoperoxidase staining was performed on the cell monolayers as described above. Cells transfected with p3CLb* plasmids and detected with antibodies specific for the expected gene product were stained. Differences in expression were seen between the different transfections, as evidenced by the overall amount of staining. Transfections and staining were performed with the p3CIa* plasmids with similar results.

References

Chan, H. W., M. A. Israel, et al. (1979). “Molecular cloning of polyoma virus DNA in Eschericia coli: lambda phage vector system.” Science 203: 887-892.

Donnelly, J. J., J. B. Ulmer, et al. (1994). “Immunization with DNA.” J Immunol Methods 176(2): 145-52.

Fazio, V. M. (1997). ““Naked” DNA transfer technology for genetic vaccination against infectious disease.” Res Virol 148(2): 101-8.

Hassett, D. E. and J. L. Whitton (1996). “DNA immunization.” Trends Microbiol 4(8): 307 12.

Hengge, U. R., E. F. Chan, et al. (1995). “Cytokine gene expression in epidermis with biological effects following injection of naked DNA.” Nature Genetics 10(June): 161-166.

Israel, M. A., H. W. Chan, et al. (1979). “Molecular cloning of polyoma virus DNA in Eschericia coli: plasmid vector system.” Science 203: 887-892.

Klinman, D. M., G. Yamshchikov, et al. (1997). “Contribution of CpG motifs to the immunogenicity of DNA vaccines.” J Immunol 158(8): 3635-9.

Krieg, A. M. (1996). “Lymphocyte activation by CpG dinucleotide motifs in prokaryotic DNA.” Trends Microbiol 4(2): 73-6.

Krieg, A. M., A. K. Yi, et al. (1995). “CpG motifs in bacterial DNA trigger direct B-cell activation.” Nature 374(6522): 546-9.

Lawson, C. M., W. Yeow, et al. (1997). “In vivo expression of an interferon-a gene by intramuscular injection of naked DNA.” Journal of Interferon and Cytokine Research 17: 255-261.

Pisetsky, D. S. (1996). “The immunologic properties of DNA.” J Immunol 156(2): 421-3.

Robinson, H. L., H. S. Ginsberg, et al. (1997). The Scientific Future of DNA for Immunization, http://www.asmusa.org/acasrc/acal.htm.

Roman, M., E. Martin-Orozco, et al. (1997). “Immunostimulatory DNA sequences function as T helper-i-promoting adjuvants.” Nature Medicine 3(8): 849-854.

Sato, Y., M. Roman, et al. (1996). “Immunostimulatory DNA sequences necessary for effective intradermal gene immunization.” Science 273(5273): 352-4.

Sun, S., C. Beard, et al. (1977). “Mitogenicity of DNA from different organisms for murine B cells.” Journal of Immunology 159: 3119-3125.

Wang, C., L. Chao, et al. (1995). “Direct gene delivery of human tissue kallilrein reduces blood pressure in spontaneously hypertensive rats.” J. Clin. Invest 95(April): 1710-1716.

Will, H., R. Cattaneo, et al. (1982). “Cloned HBV DNA causes hepatitis in chimpanzees.” Nature 299: 740-742.

Yi, A. K., J. H. Chace, et al. (1996). “IFN-gamma promotes IL-6 and IgM secretion in response to CpG motifs in bacterial DNA and oligodeoxynucleotides.” J. Immunol 156(2): 558-64.

### Example 2

### Injection of Plasmids Expressing Antigen Alone or Cytokine and Antigen Into Mice and Swine.

Naked DNA technology, or genetic immunization as it is now being called, is the spontaneous uptake and expression, by mammalian cells, of injected DNA, to produce an immune response. The technology has become very popular recently and has been applied to a wide variety of viruses as well as some bacteria and parasites (Lopez-Macias, Lopez-Hernandez et al. 1995; Yang, Waine et al. 1995; Huygen, Content et al. 1996; Tascon, Colston et al. 1996; Kurar and Splitter 1997; Lai, Pakes et al. 1997; Luke, Carner et al. 1997; Strugnell, Drew et al. 1997). Specific antibody production is almost always seen in response to the injections and is often accompanied by a CTL response. In many cases, this has led to protection, against challenge by the pathogen of interest (Robinson, Ginsberg et al. 1997).

Reports on the co-administration of cytokines and plasmid DNA suggest that it is possible to manipulate the immune response, depending on the cytokine used (Irvine, Rao et al. 1996; Ramsay and Ramshaw 1997). Other researchers have attempted to use cytokines to modify the immune response by simultaneous injection of cytokine and antigen DNA's (Stevenson, Zhu et al. 1995; Xiang and Ertl 1995) or DNA expressing antigen/cytokine fusions (Maecker, Umetsu et al. 1997). Introduction of DNA encoding cytokines or other proteins has been used for therapeutic purposes (Raz, Watanabe et al. 1993; Raz, Dudler et al. 1995; Sun, Burkholder et al. 1995; Keller, Burkholder et al. 1996; Vermeij and Blok 1996; Daheshia, Kuklin et al. 1997).

Materials and Methods

Plasmids: The plasmids used in this study are described above in Example 1. Plasmid DNA was isolated from E. coli bacteria using NaOH/SDS with subsequent purification by either CsCl gradient centrifugation or QIAGEN columns. For storage, DNA was resuspended, at high concentration, in TE (10 mM tris:0.1 mM EDTA. pH=8.0). For injection into animals, the DNA was diluted to the desired concentration in PBS.

ELISA Methods: Detection of anti-IFNK and anti-KIII was with an indirect ELISA using purified protein bound to a microtiter plate as described previously.

Animal Inoculations: Mice were pretreated with either PBS or bupivacaine (%), seven days prior to inoculation with plasmid DNA. DNA was introduced via four 25 μl injections into the quadriceps muscles.

Swine were injected with DNA, IM in the ham, using an 18 gauge needle. Various parameters were examined to determine the technique yielding the best results (FIG. 2). A single 2 ml injection of lmg of DNA at full needle depth in these studies.

Results and Discussion

Mouse Studies: Our initial investigation examining the expression, in vivo, of our constructs was conducted in mice. The primary reason for this was size. Using mice we were able to look at a greater number of parameters such as plasmids with or without introns and different amounts of DNA per injection. We were also able to evaluate reports that injection into regenerating muscle tissue results in higher gene expression normal muscle ((Acsadi, Dickson et al. 1991; Wang, Ugen et al. 1993; Danko and Wolff 1994; Wang, Merva et al. 1994)).

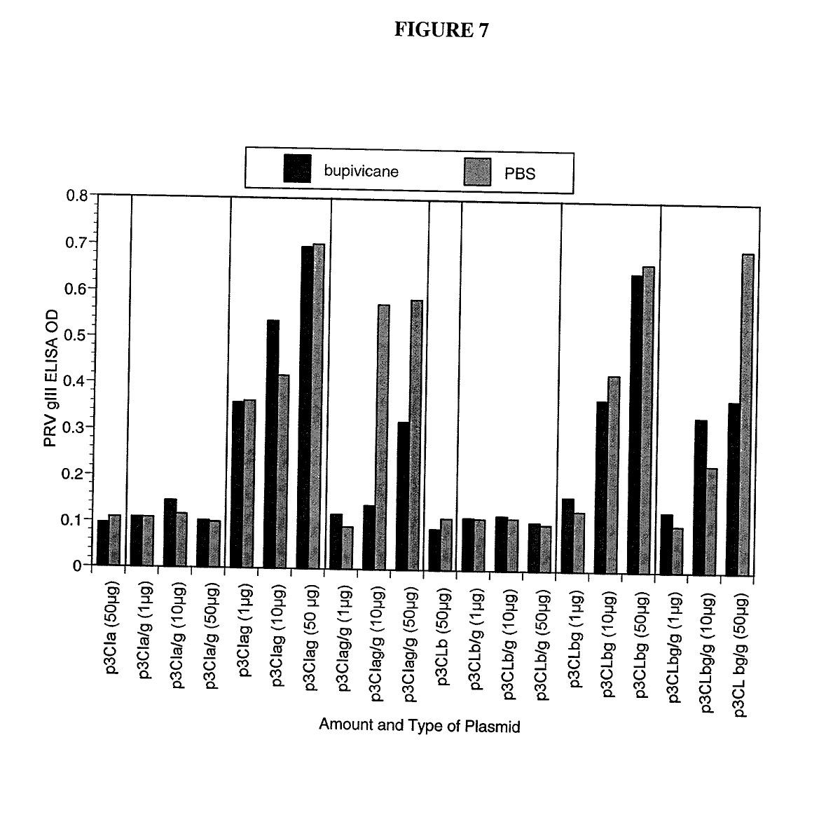

As described above, mice were pretreated, 7 days prior to injection, with PBS or bupivacaine. They were then injected with varying amounts of eight different plasmids (p3CIa, p3CIag, p3CIa/IFNg, p3CIag/IFNg, p3CLb, p3CLbg, p3CLb/IFNg, and p3CLbg/IFNg). The mice were bled 2 weeks later and antibody to PRV gIIl was detected by ELISA (FIG. 7).

Increasing amounts of DNA produced a greater immune response. We saw no difference in response depending on whether or not the plasmids contained an intron. Perhaps the most interesting result was the response to treatment with PBS or bupivacaine prior to injection. There was no marked increase in the bupivacaine group. In fact, some groups demonstrated greater response with PBS. This was unexpected and contrary to published reports. Thus, injection technique and the necessity of consistently hitting the muscle are directly related to response.

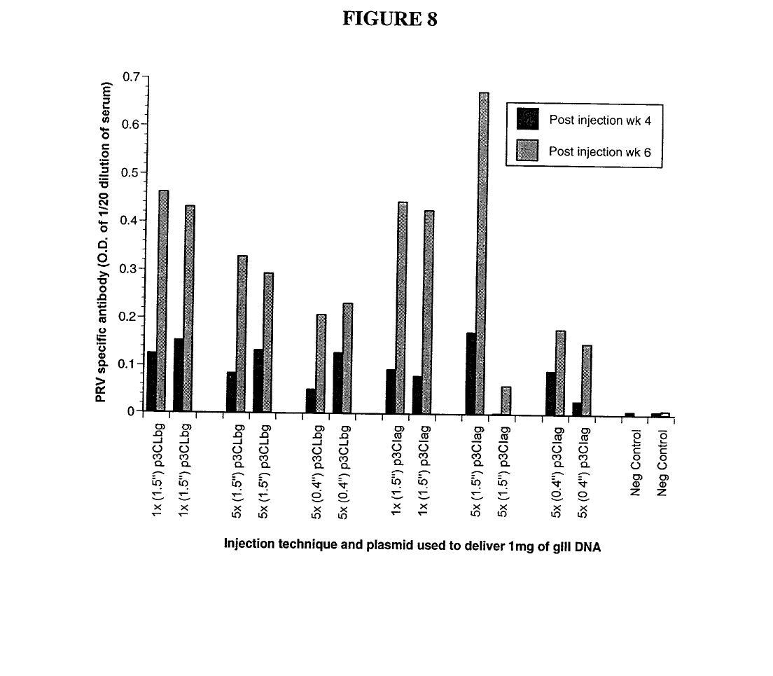

Swine Studies: A study of injection technique was done to determine if number, depth, or volume of injection had any effect on the immune response to gIII plasmid DNA. Pigs were injected with 1 mg of p3CIag or p3CLbg DNA, IM, at a single DNA concentration of 0.5 mg/ml. Multiple versus single injection at different depths of penetration and with different volumes were compared (FIG. 8). The animals received a booster inoculation after 3 weeks. The pigs were bled at 3 and 6 weeks after the initial injection and anti-gIII antibody was detected by ELISA. Final results indicated that a single, 2 ml injection, at full depth with an 18 gauge needle, produced the best results.

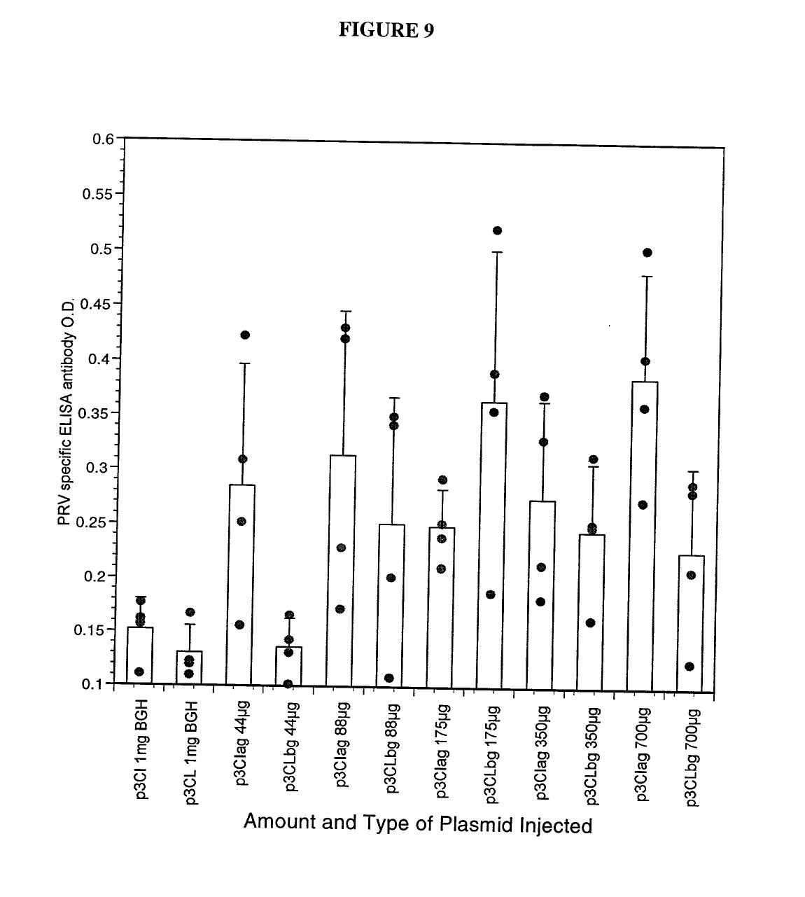

An experiment similar to that done in mice, but not as large in scope, was performed with pigs. Briefly, plasmids p3CIag and p3CLbg were injected into pigs, at 5 different concentrations, in a 2 ml volume (see above). At 3 weeks, the animals were boosted, and at 6 weeks, PRV gIII specific antibody was detected by ELISA (FIG. 9). A general trend of increasing response to increased DNA concentration was seen, however there was wide individual variability in responses to the same treatment. Again, there was no obvious effect of having or not having the intron in a construct.

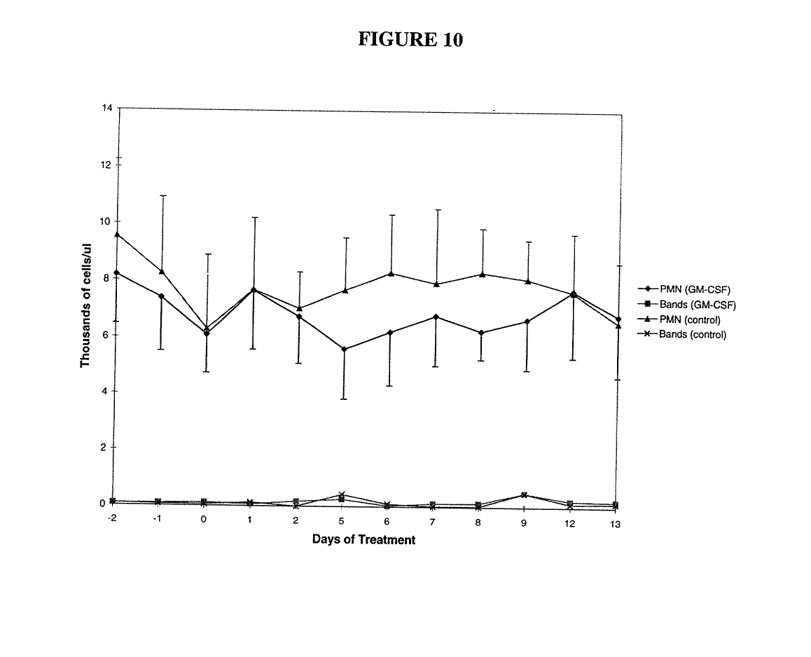

Two experiments were designed to look for a biological effect of injection with plasmid DNA carrying cytokine genes. One study involved injecting pigs with the plasmid p3CIa/GM-CSF and looking for a change in the numbers of PMN's and bands. Controls were injected with PBS alone. There were 6 pigs in each group. Pigs were bled daily and the numbers of PMN's and bands were determined. The expected result was to see an increase in PMN's in the GM-CSF group. The wide variability in individual responses did not allow any statistically significant conclusions. However, there did appear to be a slight depression in PMN's in the GM-CSF group (FIG. 10).

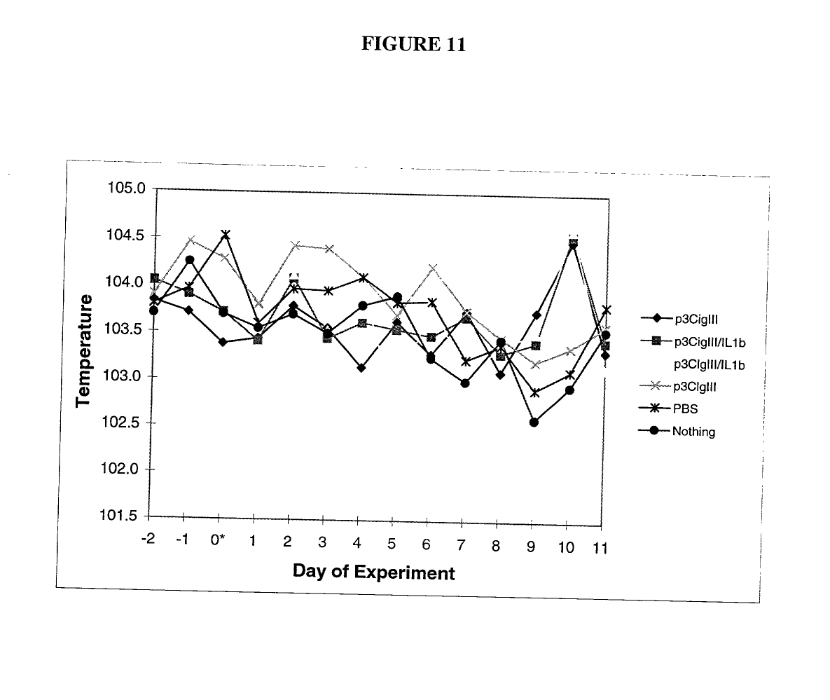

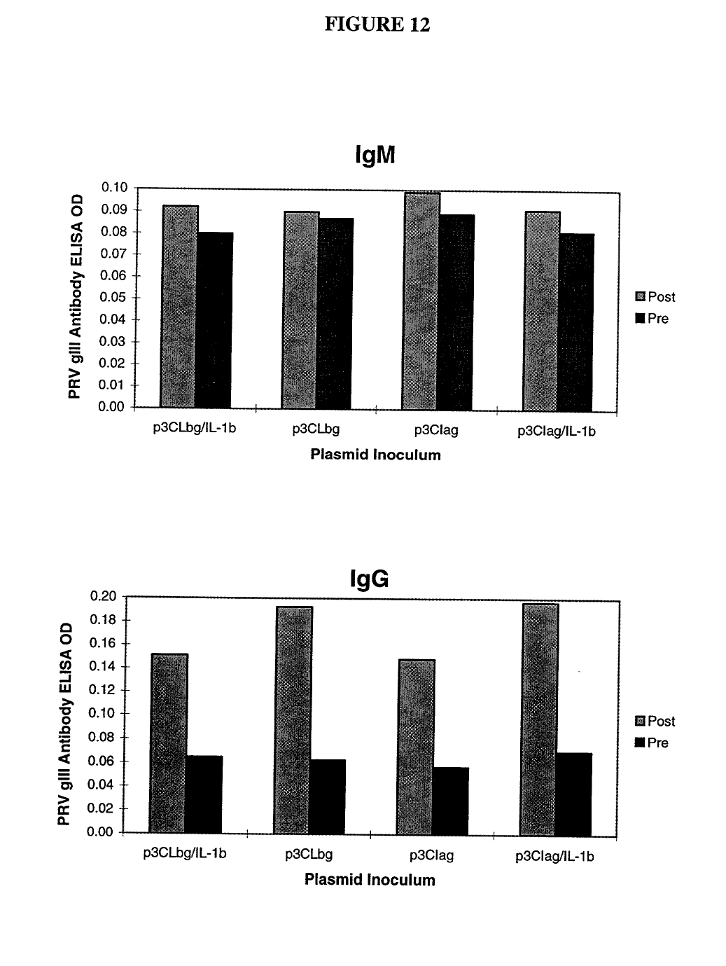

The other study compared the effect, on temperature, in pigs treated with p3CIag, p3CIag/IL-1B, p3CLbg, p3CLbg/IL-1B, and PBS. Each group contained 6 pigs, except for the uninjected group, which had 3 pigs. Pigs were temped daily for a period of 15 days and were bled on the final day of the study. To confirm expression of the chimeric gIII/IL-1B proteins, antibody specific to PRV gIII was also measured. Once again, the inconsistency of the individual responses did not allow for any meaningful conclusion regarding the possible effects of the IL-1υ DNA (FIG. 11). However, presence of PRV gIII specific antibody in all the treated groups suggests that both the gIII and the chimeric gIII/IL-1B proteins were being produced in pigs after injection with plasmid DNA (FIG. 12).

Reference

Acsadi, G., G. Dickson, et al. (1991). “Human dystrophin expression in mdx mice after intramuscular injection of DNA constructs.” Nature 352(6338): 815-8.

Daheshia, M., N. Kuklin, et al. (1997). “Suppression of ongoing ocular inflammatory disease by topical administration of plasmid DNA encoding IL-10.” J Immunol 159(4): 1945-52.

Danko, I. and J. A. Wolff (1994). “Direct gene transfer into muscle.” Vaccine 12(16): 1499-502.

Huygen, K., J. Content, et al. (1996). “hnmunogenicity and protective efficacy of a tuberculosis DNA vaccine.” Nat Med 2(8): 893-8.

Irvine, K. R., J. B. Rao, et al. (1996). “Cytokine enhancement of DNA immunization leads to effective treatment of established pulmonary metastases.” J Immunol 156(1): 238-45.

Keller, E. T., J. K. Burkholder, et al. (1996). “In vivo particle-mediated cytokine gene transfer into canine oral mucosa and epidermis.” Cancer Gene Ther 3(3): 186-91.

Kurar, E. and G. A. Splitter (1997). “Nucleic acid vaccination of Brucella abortus ribosomal L7/L12 gene elicits immune response.” Vaccine 15(17-18): 1851-7.

Lai, W. C., S. P. Pakes, et al. (1997). “Therapeutic effect of DNA immunization of genetically susceptible mice infected with virulent Mycoplasma pulmonis.” J Immunol 158(6): 2513-6.

Lopez-Macias, C., M. A. Lopez-Hernandez, et al. (1995). “Induction of antibodies against Salmonella typhi OmpC porin by naked DNA immunization.” Ann N Y Acad Sci 772: 285-8.

Luke, C. J., K. Carner, et al. (1997). “An OspA-based DNA vaccine protects mice against infection with Borrelia burgdorferi.” J Infect Dis 175(1): 91-7.

Maecker, H. T., D. T. Umetsu, et al. (1997). “DNA vaccination with cytokine fusion constructs biases the immune response to ovalbumin.” Vaccine 15(15): 1687-96.

Ramsay, A. J. and I. A. Ramshaw (1997). “Cytokine enhancement of immune responses important for immunocontraception.” Reprod Fertil Dev 9(1): 91-7.

Raz, E., J. Dudler, et al. (1995). “Modulation of disease activity in murine systemic lupus erythematosus by cytokine gene delivery.” Lupus 4(4): 286-92.

Raz, E., A. Watanabe, et al. (1993). “Systemic immunological effects of cytokine genes injected into skeletal muscle.” Proc Natl Acad Sci USA 90(10): 4523-7.

Robinson, H. L., H. S. Ginsberg, et al. (1997). The Scientific Future of DNA for Immunization, http://www.asmusa.org/acasrc/aca1.htm.

Stevenson, F. K., D. Zhu, et al. (1995). “A genetic approach to idiotypic vaccination for B cell lymphoma.” Ann N Y Acad Sci 772: 212-26.

Strugnell, R. A., D. Drew, et al. (1997). “DNA vaccines for bacterial infections.” Immunol Cell Biol 75(4): 364-9.

Sun, W. H., J. K. Burkholder, et al. (1995). “In vivo cytokine gene transfer by gene gun reduces tumor growth in mice.” Proc Natl Acad Sci USA 92: 2889-93.

Tascon, R. E., M. J. Colston, et al. (1996). “Vaccination against tuberculosis by DNA injection.” Nat Med 2(8): 888-92.

Vermeij, P. and D. Blok (1996). “New peptide and protein drugs.” Pharm World Sci 18(3): 87-93.

Wang, B., M. Merva, et al. (1994). “DNA inoculation induces protective in vivo immune responses against cellular challenge with HIV-1 antigen-expressing cells.” AIDS Res Hum Retroviruses 10(2): S35-41.

Wang, B., K. E. Ugen, et al. (1993). “Gene inoculation generates immune responses against human immunodeficiency virus type 1. ” Proc Natl Acad Sci USA 90(9): 4156-60.

Xiang, Z. and H. C. Ertl (1995). “Manipulation of the immune response to a plasmid-encoded viral antigen by coinoculation with plasmids expressing cytokines.” Inmunity 2(2): 129-35.

Yang, W., G. J. Waine, et al. (1995). “Antibodies to Schistosomajaponicum (Asian bloodfluke) paramyosin induced by nucleic acid vaccination.” Biochem Biophys Res Commun 212(3): 1029-39.

### Example 3

### Effect of Introduction of Naked DNA Encoding pST into Swine

Materials and Methods

Eighty weaned crossbred Yorkshire pigs (40 gilts and 40 barrows) were obtained from the PNU breeding herd. As the animals approached a body weight (BW) of 25-30 kg they were allotted by BW and gender into five blocks of eight pigs/gender. Within each block, two pigs/gender were assigned randomly to one of four pens/block. Pigs were allowed ad libitum access to a diet of 24% crude protein (CP) starting at the acclimation period (one week prior to administration of pST or genetic immunization). This amount of CP ensured that adequate amino acid was available in case there of a a reduction in voluntary feed intake caused by exogenous pST treatment. After a 7-d acclimation period the pigs were weighed (day 0) and then each pen of the animals within a block were either noninjected (control) or subjected to 42 daily i.m. injections of 1-2 mL of saline containing 60 μg/mL of recombinant pST/kg BW, three bi-weekly i.m. injections of 2 mL saline containing 1 mg naked plasmid pST-DNA or five weekly i.m. injections of 2 mL saline containing 100 ug of naked plasmid pST-DNA+8ug of bupivicaine. The latter formulation was intended to take advantage of a newly reported phenomenon in which low concentrations of bupivicaine spontaneously forms liposomes upon mixing with DNA.

Treatment pens within a block were assigned randomly on the first day of treatment. Average daily gain (ADG) of pigs were determined weekly. Feed utilization was determined by the difference in feed weight at the end of the trial versus start of trial plus feed added. The ratio of feed utilization:BW gain represented feed efficiency.

The study was terminated after thirteen weeks as most animals had reached a marketable weight of 110-120 kg. The barrows within the control groups and the naked DNA groups were analyzed for carcass quality by measuring back fat thickness at the first, 10 and last lumber ribs, and the area of the loin eye between the 10^{th} and 11^{th} ribs. Results are shown in Table 1.

Results

Both DNA groups showed increased body weight gains when compared to controls for the first 10 weeks of the study. From the eleventh week, they maintained their previously obtained body weight advantage, but they did not show an improvement in performance when compared to controls.

No obvious change in feed efficiency existed when compared to controls. Thus they ate the same amount of food normal pigs would to make such body weight changes. Daily pST administration may cause suppression of appetite, and as the DNA pigs did not eat less their levels of pST could not be close to that induced by the injection of 60μg/kg pST protein.

No change in carcass composition between controls and the naked DNA groups was apparent. This could be interpreted in several ways. It seems that the naked DNA groups did not show a difference in growth patterns during their last three weeks. If the naked DNA stopped producing then the pigs would quickly have reverted to the lean and fat distribution of a normal pig. Thus it is possible that the expected changes of increased lean and less fat did exist after 10 weeks. Alternatively, it is possible that expression of pST did not diminish, but rather the pigs stopped responding to the low amounts of pST produced by the naked DNA at this stage of maturation ( a phenomenon seen in other species towards the end of their growing phase).

Thus, the naked DNA made sufficient pST for the first 10 weeks so as to increase body weight gain over controls by approx 2-5%. The advantage of this would be that pigs could be expected to get to market about 5-7 days earlier than expected, leading to savings in facility and labour costs.

It will be clear that the invention may be practiced otherwise than as particularly described in the foregoing description and examples.

Numerous modifications and variations of the present invention are possible in light of the above teachings and, therefore, are within the scope of the invention.

The entire disclosure of all publications cited herein are hereby incorporated by reference.

TABLE 1
BODY WEIGHT GAINS

| Day | Control | Control stdevs | 60 μg/kg r-pst | r-pst stdevs | 1 mg pst DNA | 1 mg stdevs | 100 μg pst DNA | 0.1 mg stdevs |
|---|---|---|---|---|---|---|---|---|
| 0 | 33.1 | 2.78 | 33.98 | 2.86 | 33.94 | 3.42 | 34.32 | 3.06 |
| 7 | 39.2 | 3.12 | 40.78 | 3.26 | 40.45 | 2.76 | 40.00 | 2.59 |
| 14 | 45.5 | 3.30 | 47.84 | 3.11 | 46.60 | 3.16 | 46.68 | 2.74 |
| 21 | 51.0 | 3.61 | 54.29 | 2.18 | 52.31 | 3.10 | 52.52 | 2.89 |
| 28 | 57.5 | 3.92 | 62.05 | 2.18 | 59.49 | 3.29 | 59.52 | 3.36 |
| 35 | 63.9 | 4.09 | 69.70 | 2.01 | 65.85 | 3.50 | 65.8 | 3.06 |
| 42 | 70.6 | 3.44 | 77.96 | 2.86 | 72.94 | 3.97 | 73.0 | 3.0 |
| 49 | 76.6 | 3.78 | 80.62 | 3.03 | 78.81 | 4.33 | 78.93 | 3.01 |
| 56 | 81.5 | 3.78 | 85.59 | 3.30 | 84.89 | 4.46 | 85.11 | 2.85 |
| 63 | 88.7 | 4.07 | 91.6 | 2.84 | 91.31 | 4.31 | 92.60 | 3.21 |
| 70 | 95.4 | 4.85 | 99.22 | 3.75 | 98.77 | 4.68 | 99.29 | 3.22 |
| 77 | 101.2 | 4.43 | 104.76 | 3.66 | 104.56 | 4.50 | 105.42 | 2.57 |
| 84 | 108.8 | 4.70 | 112.19 | 3.54 | 111.34 | 4.30 | 112.25 | 3.45 |
| 91 | 115.6 | 3.33 | 118.15 | 3.93 | 117.93 | 5.63 | 118.18 | 3.73 |

What is claimed is:

1. A method for delivering a desired physiologically active protein, polypeptide or peptide growth hormone or cytokine to a non-human vertebrate, comprising injecting into the muscle of said vertebrate a non-infectious, non-immunogenic, non-integrating DNA sequence encoding said growth hormone or cytokine operably linked to a promoter, wherein said DNA sequence is free from association with transfection-facilitating proteins, viral particles, liposomal formulations, charged lipids and calcium phosphate precipitating agents, whereby said DNA sequence is expressed and induces an increase in body weight gain in said vertebrate.

2. The method of claim 1 wherein said vertebrate is a mammal.

3. The method of claim 1 wherein said growth hormone or cytokine is selected from the group consisting of porcine growth hormone, bovine growth hormone, canine growth hormone, bovine IGF-1, porcine IGF-1, canine IGF-1, bovine growth hormone releasing factor, porcine growth hormone releasing factor, and canine growth hormone releasing factor.

4. The method of claim 3, wherein said growth hormone or cytokine has an amino acid sequence identical to the native growth hormone or cytokine of said vertebrate.

5. The method of claim 4, wherein said growth hormone or cytokine is porcine growth hormone, and said vertebrate is a pig.

6. The method of claim 4, wherein said growth hormone or cytokine is bovine growth hormone, and said vertebrate is a cow.

7. The method of claim 4, wherein said growth hormone or cytokine is canine growth hormone, and said vertebrate is a dog.

8. The method of claim 4, wherein said growth hormone or cytokine is bovine IGF-1, and said vertebrate is a cow.

9. The method of claim 4, wherein said growth hormone or cytokine is porcine IGF-1, and said vertebrate is a pig.

10. The method of claim 4, wherein said growth hormone or cytokine is canine IGF-1, and said vertebrate is a dog.

11. The method of claim 4, wherein said growth hormone or cytokine is bovine growth hormone releasing factor, and said vertebrate is a cow.

12. The method of claim 4, wherein said growth hormone or cytokine is porcine growth hormone releasing factor, and said vertebrate is a pig.

13. The method of claim 4, wherein said growth hormone or cytokine is canine growth hormone releasing factor, and said vertebrate is a dog.

14. The method of claim 1, wherein said DNA is free from a delivery vehicle to facilitate entry of the DNA into the cell.

15. The method of claim 1, wherein said DNA comprises a plasmid.

16. The method of claim 1, wherein said promoter is a cell specific or tissue specific promoter.

17. The method of claim 1, wherein said muscle is skeletal muscle.

18. The method of claim 1, wherein said injection comprises impressing said DNA through the skin.

19. The method of claim 1, wherein said injection comprises injection of said DNA through a needle.

20. The method of claim 1, wherein said injection comprises injecting said DNA into the interstitial space of said muscle resulting in transfection of said DNA into muscle cells of said vertebrate.

21. The method of claim 1, wherein said DNA is operably linked to a DNA sequence encoding a signal peptide wherein the signal peptide directs the secretion of the growth hormone or cytokine.

22. The method of claim 1, where the expression of said growth hormone or cytokine is transitory.

23. The method of claim 1, wherein said growth hormone or cytokine is produced for at least one month.
